(19) United States
(12) Patent Application Publication
(10) Pub. No.: US 2020/0066390 A1
(43) Pub. Date: Feb. 27, 2020

(54) Physical Therapy System and Method

(71) Applicant: Verapy, LLC, Tampa, FL (US)

(72) Inventors: Tad Svendrys, Tampa, FL (US); Jonathan Truong, Tampa, FL (US)

(73) Assignee: Verapy, LLC, Tampa, FL (US)

(21) Appl. No.: 16/539,952

(22) Filed: Aug. 13, 2019

Related U.S. Application Data
(60) Provisional application No. 62/720,426, filed on Aug. 21, 2018.

(51) Int. Cl. G16H 20/30; G06F 3/01

(52) U.S. Cl. CPC G16H 20/30; A63B 71/0622; G06F 3/012; G06F 3/016

(57) ABSTRACT

A Physical Therapy Occupational Therapy (PT/OT) system, method, and environment using game embodiments is disclosed. The game embodiments are created by qualified astute and assertive game developers, and thus are games first and foremost. This results in the games having a higher level of entertainment value, yet still providing important PT/OT functionality. The embodiments transform standard PT/OT exercises into fun and immersive virtual reality games which increases efficacy, patient retention, and value to physical therapists. The embodiments also enhance patient experience, ultimately resulting in better patient outcomes. Consequently, the embodiments drive up patient retention and reduce cancellations/no shows while stabilizing clinic cash flow.

### BACKGROUND OF THE INVENTION

Today's physical and occupational therapy rehabilitation model has challenges with empowering patients to participate in their treatment. Patient non-compliance equally affect all parties involved in the healthcare community including therapists and medical payers alike. The underlying challenge is that therapy is rigorous and made up of a series of repetitive exercises. The overwhelming response from the physical rehabilitation professionals support the premise that the lack of patient compliance and the lack of engaging modalities lead to poor outcomes.

At present what few Physical and Occupational Therapy (hereinafter, PT/OT) games exist are created by healthcare professionals themselves, and are not created by experienced game developers. Instead, the games are created by including but not limited to primary care, chiropractors and other healthcare professionals so that the games may be weak, boring, low quality, and not a true game experience. That may be fine for an 85-year-old or 75-year-old World War II veteran. However, a 30-year-old who was in a car accident, and grew up on World of Warcraft® or Call of Duty®, that arrangement won't work.

Consequently, there is a desire for an improved PT/OT system that will lower the non-compliance rates, increase provider efficiency and decrease the cost of care.

### SUMMARY OF THE EMBODIMENTS

As stated, existing PT/OT systems have various limitations. In stark contrast, the PT/OT system(s) discussed herein address this by using qualified astute and assertive game developers. The game embodiments within the embodiments herein are games first and foremost, and thus higher level of entertainment value, but with a physical therapy tie-in. The efficacy, customer retention, and value increases based on the proposed physical and occupational therapy platform that turns standard exercises into fun and immersive virtual reality games. The tool is designed to enhance patient experience ultimately resulting in better patient outcomes. Consequently, the platform drives up patient retention, reduces cancellations/no shows while stabilizing clinic cash flow.

In playing the physical therapy games\exercises described herein, the patients may be collecting and unlocking “achievements,” e.g. simple things like integrating their completed therapy for the first week, to playing the first game. This will achieve a type of effect like a “self-high-five” and make them feel good like they are accomplishing something. The idea is to motivate the patient through the form of Gamification. A known problem with patients not doing their physical therapy is that some patients expect immediate results, and when they do the exercises and don't immediately feel better, they either stop, slow down a lot, or just lose interest. While loss of interest can be the patient's fault, these achievements can help overcome the loss of interest a patient might typically experience.

One way to achieve this feel-good effect is by tapping into a patient's pre-existing enjoyment of video games (where appropriate). The system 100 is usable by any/all types of patients, but the expected “sweet spot” or demographic of maximum utilization will be those individuals with a pre-existing enjoyment of video games, particularly video gamers which use VR systems.

In an embodiment, the system 100 makes use of roadmaps. A roadmap should aim for getting patients back to doing what people enjoy. Let's say someone with a wrist injury wants to (eventually) get back to playing tennis. The first thing with wrist injuries must be restoring the ability to pick up a coffee cup, based on day to day life experiences and activities of daily living (ADLs). The purpose is to achieve patient empowerment, to see the result, but also continue that therapy day in and day out, without lapses or blow offs.

Within all embodiments disclosed herein, the gamification aspect a key component. The video game market is big. A lot of people receiving physical therapy are familiar with and sometimes enjoy video games. Accordingly, the system 100 might borrow from, imitate, or incorporate patient-input and GUI features from popular games, to increase patient-assimilation. Such a familiarity aspect could work to take some of the dullness out of the physical therapy routines (exercises), arguably providing an increased motivation level. Many patients do not want to do their physical therapy, do not enjoy it, and yet are aware of the benefits, at least in an intellectual sense. However, such patients may in the past still blew off their PT, but now, with the system 100, make it part of something they regularly do anyway (e.g. play video games). As such, adding an entertainment aspect, to what may already be a recognized gaming routine in their life, might lead to a higher level of patient attitude, efficacy, and a higher level of patient compliance.

### BRIEF DESCRIPTION OF THE DRAWINGS

FIG. 1 shows an example system incorporating the embodiments herein;

FIG. 2A shows a path of choices showing how a physical therapist or another medical advisor will choose a plan of treatment using a secure web interface hosted on the virtual service component;

FIG. 2B shows how the system processes patient choices in setting up the various games and accommodating the patient's requirements;

FIG. 3 shows a patient using an example arrangement of VR equipment, comprising at least a VR headset and body attachment.

FIGS. 4A and 4B show example GUIs displayed to new patients when they are first encountering the embodiments herein, including for example creating a patient profile as well as a potential plan of care;

FIGS. 5A and 5B show some example, non-limiting GUIs used by a physical therapist, including mechanisms enabling the physical therapist to communicate directly with patients;

FIG. 6A shows where a patient will be prompted to enter a pain level before starting a game session, while FIG. 6B shows a GUI displayed when that same patient completes the full session and are asked about pain level again;

FIG. 7A shows a GUI presented to a potential patient, showing how a game can have three options e.g. beginner, intermediate, advanced, while FIG. 7B shows an example menu displaying various options to a patient;

FIG. 8 shows an example-only, non-limiting GUI that a patient would encounter upon first logging into the embodiments;

FIG. 9A shows an example-only, non-limiting GUI of potential body-areas needing PT/OT;

FIGS. 9B and 9C show example-only, non-limiting library lists of various PT/OT games, while FIGS. 9D and 9E show GUIs of example, non-limiting game-environments as seen by a patient;

FIG. 9F shows what game-GUI would appear to a patient supposing the patient selected the sand-castle game from the GUI of FIG. 9C;

FIGS. 10A and 10B show example non-limiting GUI screen captures of potential ways of displaying patient data;

FIGS. 11A, 11B, and 12 show example, non-limiting GUI of a therapist dashboard, including example patients;

FIGS. 13A and 13B show example, non-limiting GUIs displaying patient pain levels;

FIGS. 14A and 14B show example, non-limiting GUIs for obtaining range-of-motion levels from patients, while FIGS. 14C, 14D, and 14E show example, non-limiting GUIs for displaying those range-of-motion levels;

FIGS. 15A, 15B, 15C, and 15D show example, non-limiting GUIs for patient-tutorials showing patients how to use the embodiments herein; and

FIG. 16 shows an example, non-limiting GUI for alerting a patient to a potential error or disconnection of a sensor.

### DETAILED DESCRIPTION OF THE PREFERRED EMBODIMENTS

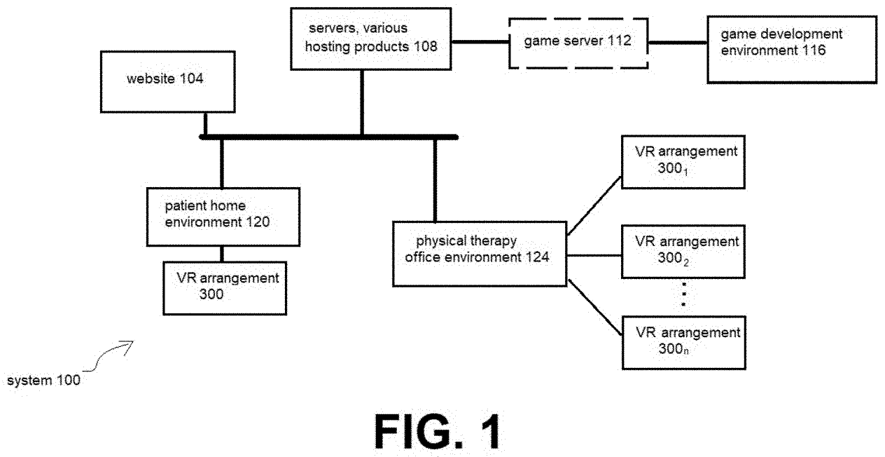

FIG. 1 shows an example system 100, comprising a therapist and patient web access portal 104, an arrangement of servers, databases and cloud-based HIPAA compliant hosting services 108, (in an embodiment) an optional arrangement of game servers 112, a game development environment 116, a patient home environment 120, and a PT/OT office environment 124. The patient home environment 120 has a least one arrangement 300 of Virtual Reality (hereinafter, VR) equipment. The PT/OT office environment 124 is shown with a plurality 1 . . . N arrangements 300 of Virtual Reality equipment.

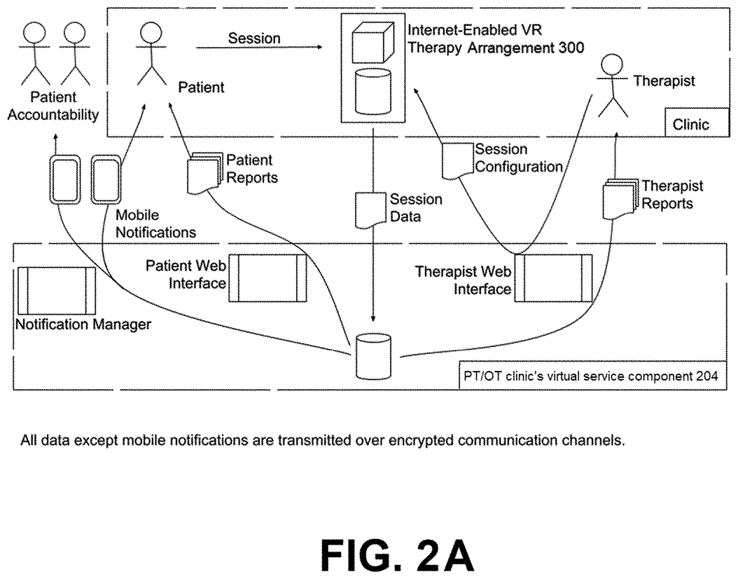

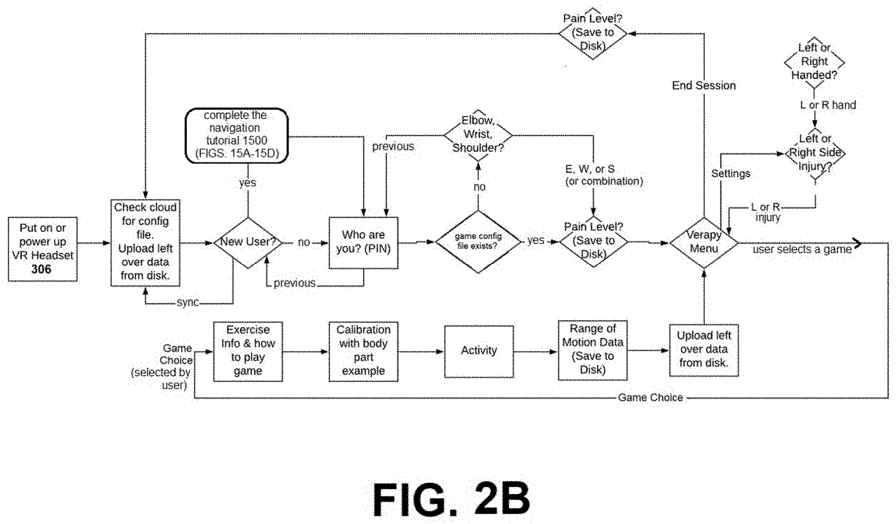

FIG. 2A shows an environment in which the physical therapist or another medical advisor will choose a plan of treatment using a secure web interface hosted on the virtual service component 204. The Virtual Service component will convert the plan of treatment into a session configuration. After that either the Internet-Enabled VR therapy arrangement 300 will download the session configuration, or, the web software will upload the session configuration to the Internet-Enabled VR therapy arrangement 300.

In an embodiment, the patient will play games using the Internet-Enabled VR Therapy arrangement 300 as part of the plan of treatment. During a treatment session, which can include one or more game play sessions, data such as pain level will be acquired directly from the patient in the form of questionnaires and data such as range of motion will be acquired indirectly and objectively from the patient based on sensor readings from the Internet-Enabled VR therapy arrangement 300.

Data recorded from a patient's therapy session (referred herein as session data) will be uploaded by the Internet-Enabled VR therapy arrangement 300 to the virtual service component 204. The Virtual Service component will generate reports, charts, and graphs for each patient based on that patient's session data to provide feedback about therapeutic progress towards goals set forth by the patient, physical therapist, medical advisor, and/or insurance company. This progress will be made available, via a web interface dashboard hosted on the Virtual Service component, to both the patient and either the physical therapist or medical advisor. Each session or multiple session data based on any specific filtering parameter is available for viewing and is exportable in e.g. a PDF format, although other data format that interface with other types of software product can also be included. Lastly, the generated report can be applied for patient billing and reimbursement claims for Medicare, private payers and such.

The patient will supply accountability contacts either to the virtual service component 204 through a web interface or to the therapist or medical advisor who will supply the contacts to the Virtual Service component. Various progress milestones will be tracked, including, but not limited to, completion of each session from the plan of treatment on schedule. If progress is not satisfactory, or if sessions from the plan of treatment are missed, then notifications will be sent to the patient and the patient's accountability contacts for motivation. These notifications may be sent via SMS, email, Android App, iPhone App, and/or other methods. Accountability contacts are typically someone with a personal relationship with the patient. However, the accountability information and patient information can include insurance companies, workman's compensation review committees, and other areas which will contribute to reducing overall health care costs.

The web interfaces of the virtual service component 204 will encrypt data in transit by secure technologies such as Secure Socket Layer (SSL). Session configuration may be encrypted on the virtual service component 204, transmitted to the internet-enabled VR therapy arrangement 300 via SSL, or both. Session data collected by the internet-enabled VR therapy arrangement 300 may be encrypted on the internet-enabled VR therapy arrangement 300, transmitted to the virtual service component 204 via SSL, or both. Data stored by the virtual service component 204 will be encrypted at rest using secure technologies such as NIST's Advanced Encryption Standard with 256-bit keys (AES-256).

Access to the web interfaces of the virtual service component 204 will be granted based on individual-level authentication using a combination of patient-name and password. The virtual service component 204 will restrict access to a patient's session configuration, session data, and reports, charts, and graphs generated from such configuration and data to the patient and physical therapists or medical advisors who are authorized to view such material.

FIG. 2B shows how the system 100 processes patient choices in setting up the various games and accommodating the patient's requirements. Within FIG. 2B, activity moves largely from left to right, although eventually returning back to the left side. A first decision is whether a configuration file already exists, e.g. on the cloud. If not, and its determined that a new user has been encountered, they are branched into a tutorial, and asked a lot of questions about the nature of their injury. Based on this information, that user will eventually be offered a choice of which game they want to play (engage with), and if they have more than one physical condition, which condition they want to address.

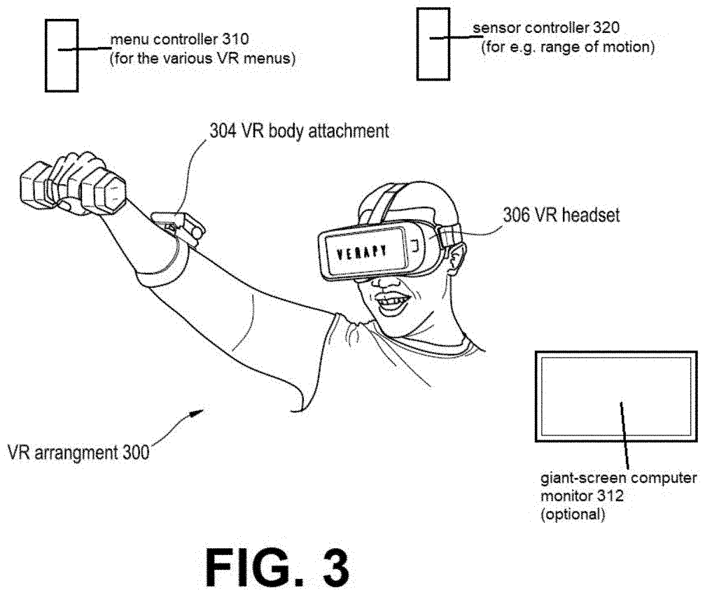

FIG. 3 shows a patient using an example arrangement 300 of VR equipment, comprising at least a VR headset 306 and a single body attachment 304, although the embodiments herein could include more than one body attachment 304₁₋ₙ. The embodiment of FIG. 3 is intentionally neutral, that is, could be implemented within either a home environment 120 or a PT office environment 124. Either way, the patient in FIG. 3 is shown doing arm exercises using the various components of an example system 100. The patient's injury may be their arm, elbow, shoulder, or other. The main visual point is that the PT of FIG. 3 is occurring somewhere within the patient's right arm.

FIG. 3 also shows a menu controller 310, for navigating through the various VR menus within the system 100, as well as potentially other purposes. FIG. 3 also shows a giant-screen computer monitor 312, in that not everyone can wear the VR headset 306. Some patients may have difficulty with VR in general, e.g. motion sickness, vertigo, or other issues related to eyesight and balance. Such patients may be precluded from using the VR headset 306, but still achieve useful results with the computer monitor 312.

Patients, therapists, and administrators can all separately log into the system 100, albeit in different contexts. There can be, for example, separate login mechanisms. The system 100 comprises both information and supervisory portals (e.g. patient portal, therapist portal, administrator portal), as well as gaming mechanisms for patient usage and therapist observation.

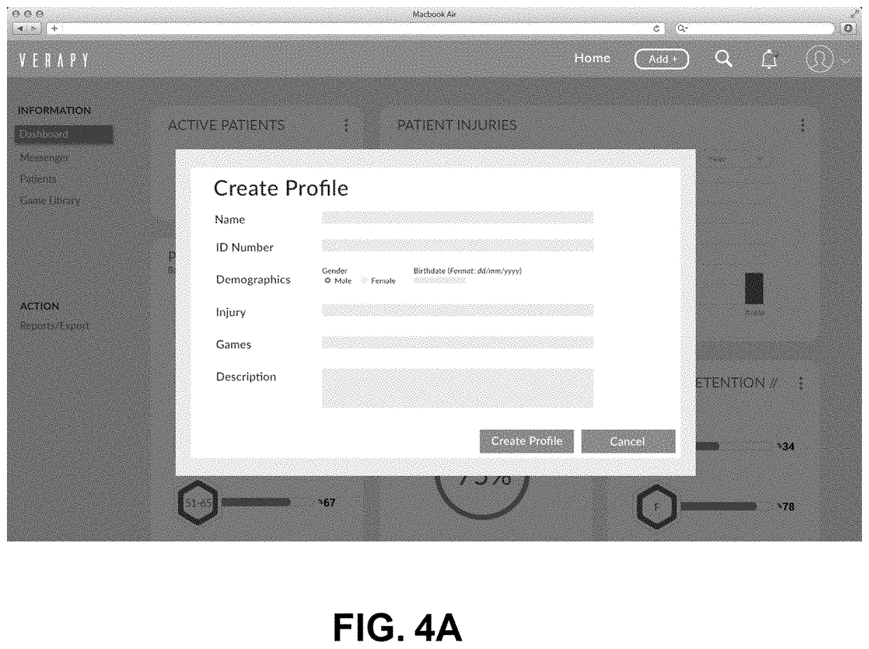

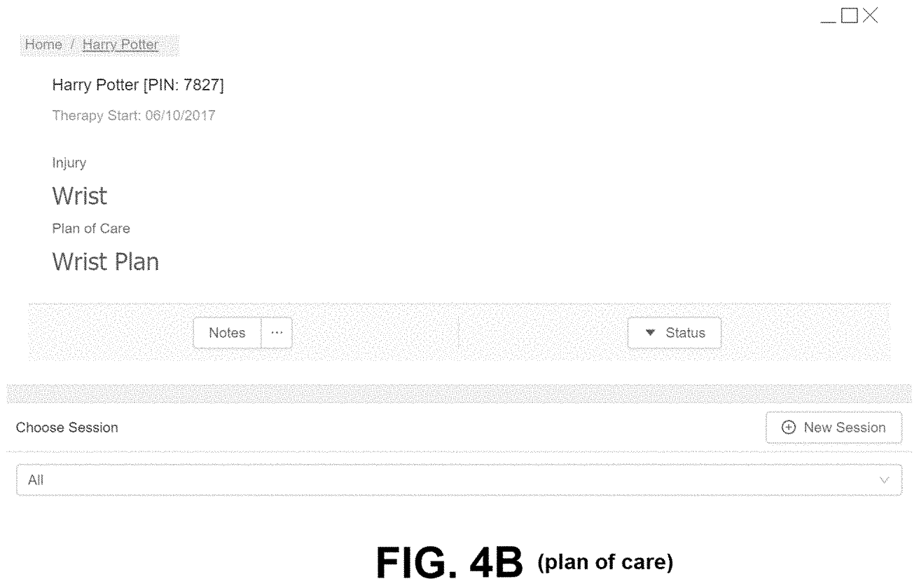

A patient must be logged into the website to access data, otherwise, the patient will be brought to the login page e.g. FIGS. 4A, 4B. The patient will be routed into a dashboard where that therapist will see the total occupation. The system 100 displays, for example, the medications prescribed, types of injury, even patients by exercise, and muscle groups.

The wearable (body attachment) devices 304₁₋ₙ. (FIG. 3) are strapped onto body parts since it only can use upper extremities, e.g. separate extremities from upper and lower. They were able to do upper and lower, thus opening up to the different kinds of injuries. The embodiments of wearables 304 could possibly include FitBit®, Apple Watch®, or even a custom device that has an accelerometer and/or a gyroscope in a sleeve or a cloth. The wearable 304 may be cotton, include Velcro, Neoprene combined with anti-microbial materials that are conducive within the healthcare and clinical environment. The wearable may also have an integrated sensor sewn inside while using the therapy program activities prescribed by the doctor e.g. keep track of the patient, doing normal walking around the house, walking the dog. In an embodiment, these non-therapy tasks may also be part of the data set of the system 100, thus incorporating activities of daily living.

FIGS. 4A and 4B show example GUIs displayed to new patients when they are first encountering the embodiments herein, including for example creating a patient profile as well as a potential plan of care. When operating the GUIs within the system 100, all patients must enter a unique patient pin code, which is a system generated unique pin code upon the creation of the new patient profile. All patient data is private and protected accessible only by their therapist and appropriate staff members. The embodiments herein thus remain HIPAA-compliant.

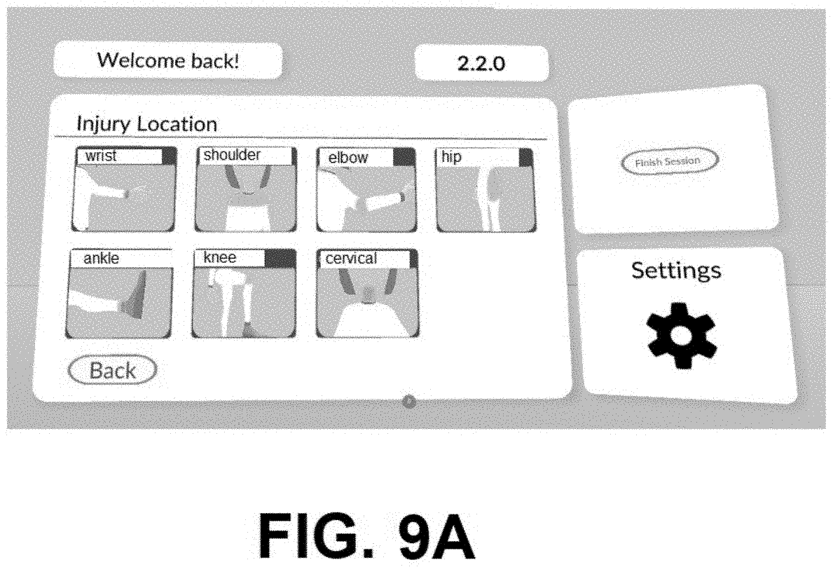

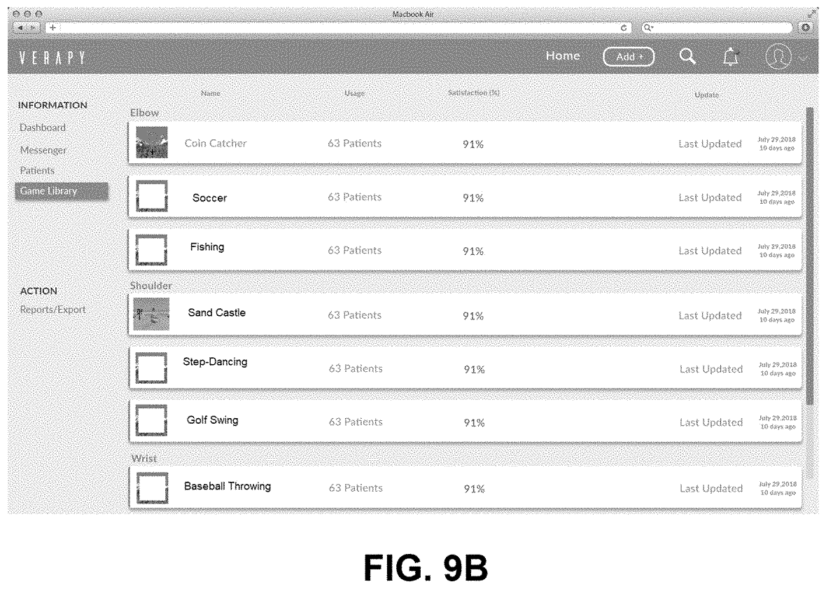

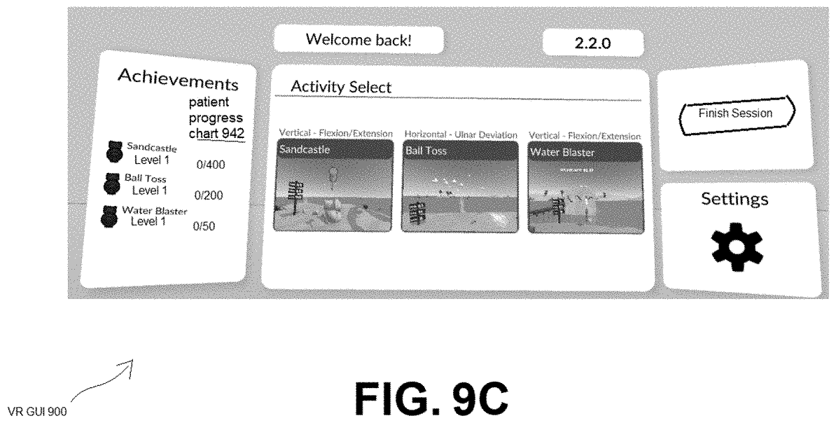

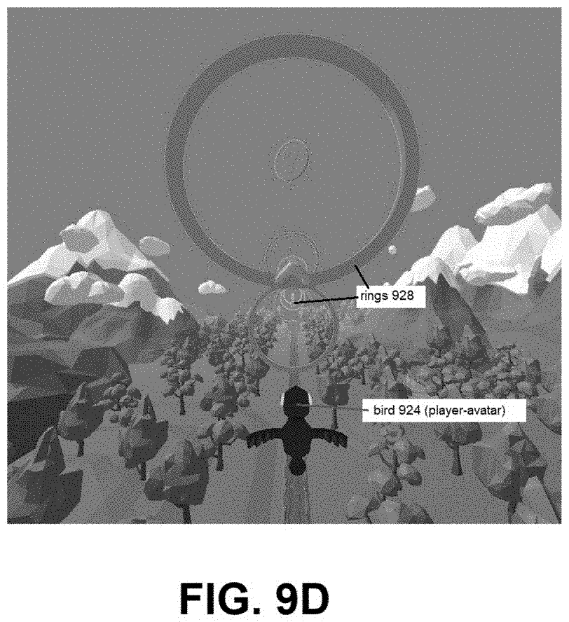

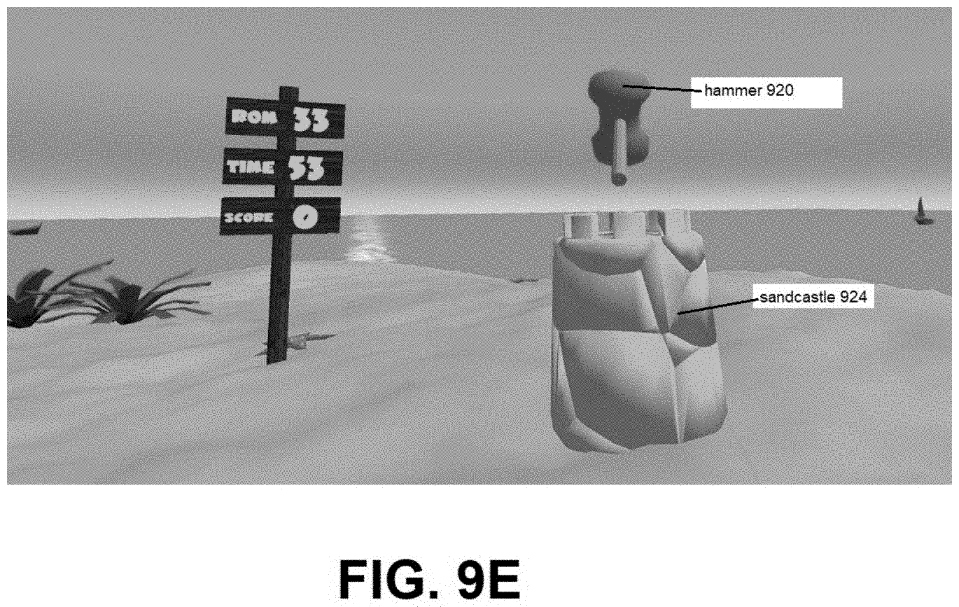

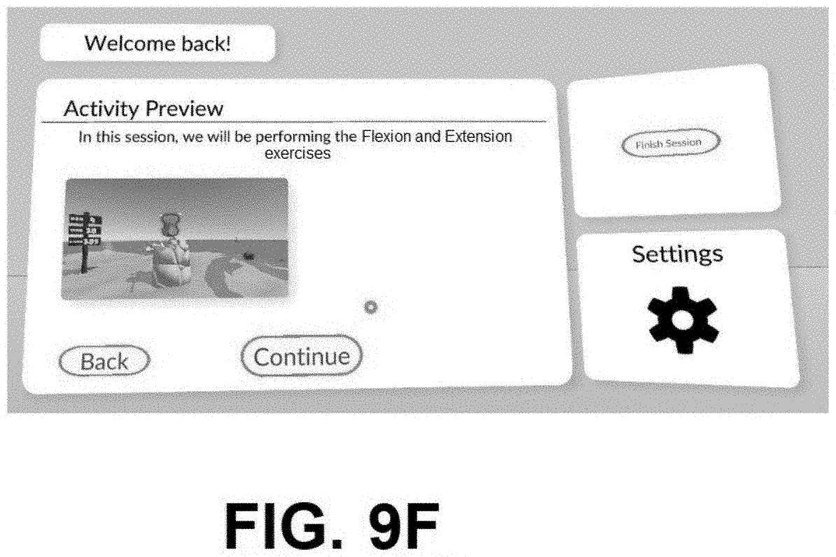

The system 100 can, if needed, display patients grouped by game, meaning all the different patients with different disabilities that play a specific injury-appropriate game. For example, one patient may achieve their best physical improvement by playing a throwing game, while another patient might play a kicking game, so a GUI (e.g. the GUI shown in FIG. 9A) will show the different injury, e.g. by ankle, shoulder, wrist, thigh, or whatever injuries that are significant.

As alluded to earlier, FIGS. 4A and 4B show GUIs of the system 100 as seen by a patient, including such metrics as the amount of progress that a patient has achieved. In an embodiment, the GUI shows progress from models are completed the time spent on one of the needs (remaining steps) to get the progress complete other therapies completed the games and injuries that corresponds with that particular patient. Once a patient goes through the “patient profiling” portion, they can view a progress chart, which is broken into at least two pieces: range of motion, and pain level. The range of motion is something whereas patients execute the games and the system 100 collects real-time data on for example the patient's progress throughout the therapy journey. A typical physical therapy patient starts out a first immediate goal, to increase their range of motion.

In an embodiment of the system 100, an ultimate goal is also shown. This is where they can consider themselves to be fully restored to optimal condition as they were before the incident or problem that necessitated physical therapy. An estimate is a set standard for where the patient should be.

A top line goal which is fully functional, full motion and zero pain, e.g. restored back to normal. An interval goal is where patients want to be at a certain time, marked by progress points, that is, points at a certain time in the future. Next, “Current” is, as expected, where that patient exists currently.

A physical therapist can use the system 100 to map out reasonable progress goals. One checkpoint might be e.g. 50% range of motion; the next week get 75% range of motion, then 100% range of motion. Another checkpoint might be a further increase in the range of motion combined with 25% strength. Looking at the injured person v. uninjured with full range of motion and, e.g. 50-75% strength.

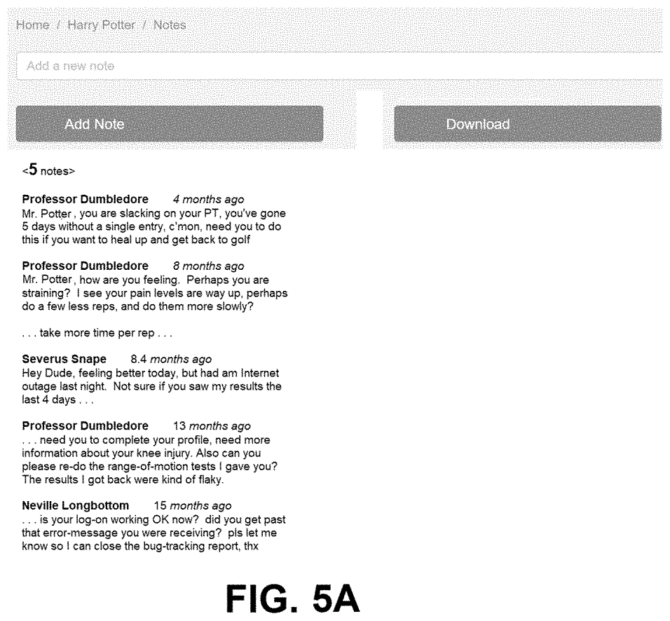

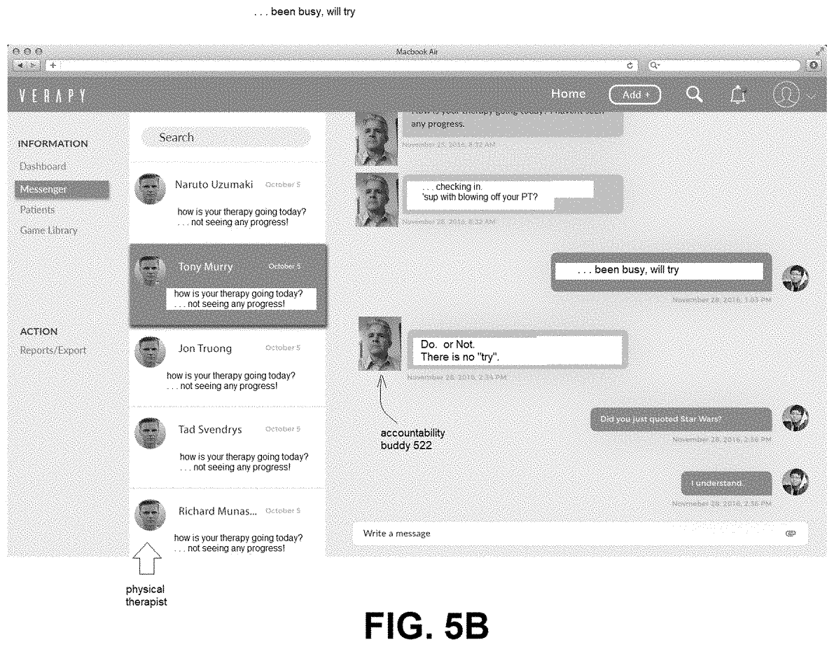

FIGS. 5A and 5B show some example, non-limiting GUIs used by a physical therapist, including mechanisms enabling the physical therapist to communicate directly with patients. In an embodiment, these communications kind look kind of like an Instant Messaging (IM) platform, although other styles can also be used. FIG. 5B shows the accountability buddy 522, a person to help prod or induce reluctant patients to step up to their PT/OT obligations, regardless of whether they are in the mood or not.

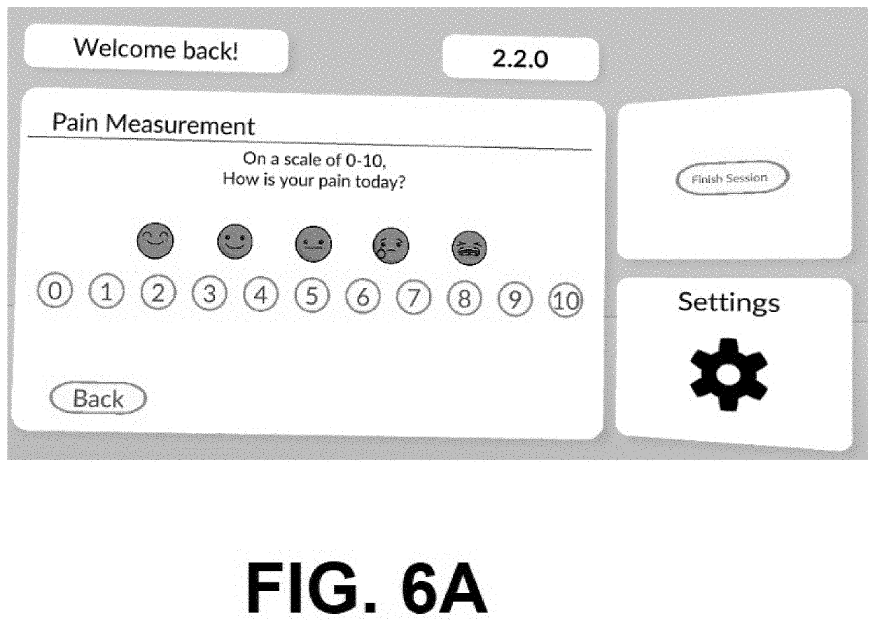

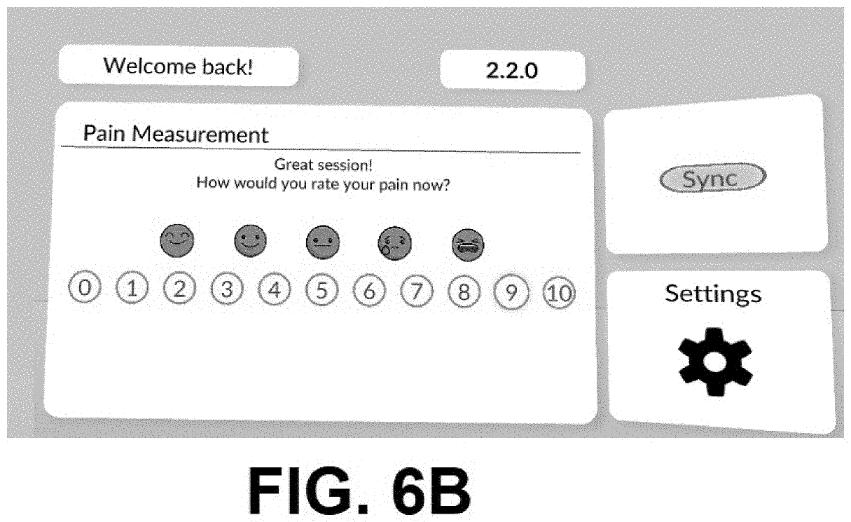

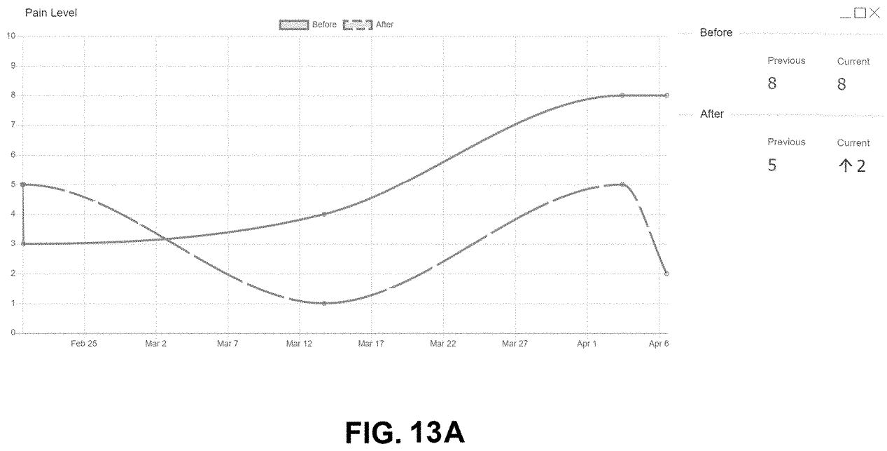

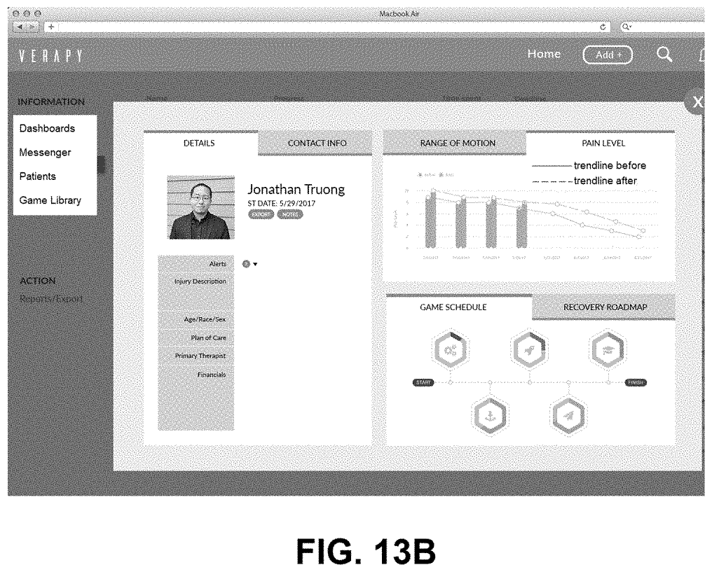

As shown in FIG. 6A, a patient will be prompted to enter her pain level on scale 0 to 10 (0 being as no pain and 10 as very painful) before starting a game. When that same patient completes their VR game session they are asked again, what is their post-game session pain level from 0 to 10, as shown in FIG. 6B. These pain levels are stored and can be mapped in a type of progression, week over week, month over month, as shown in FIGS. 13A and 13B which show example, non-limiting GUIs displaying patient pain levels. Pain level is important measurement of progress. The system 100 provides GUIs which may inquire, e.g. “is your pain increasing, decreasing, same.” Maybe a range of motion is increasing, but there is still something going on regarding pain. The presentation of objective data can demonstrate positive progress even when a flareup is causing a temporary setback.

### Begin Patient Portal

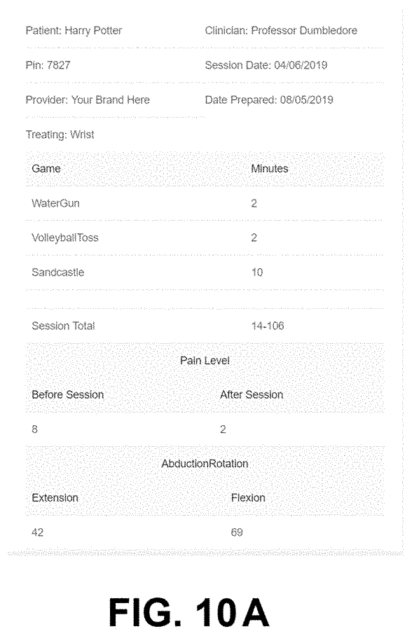

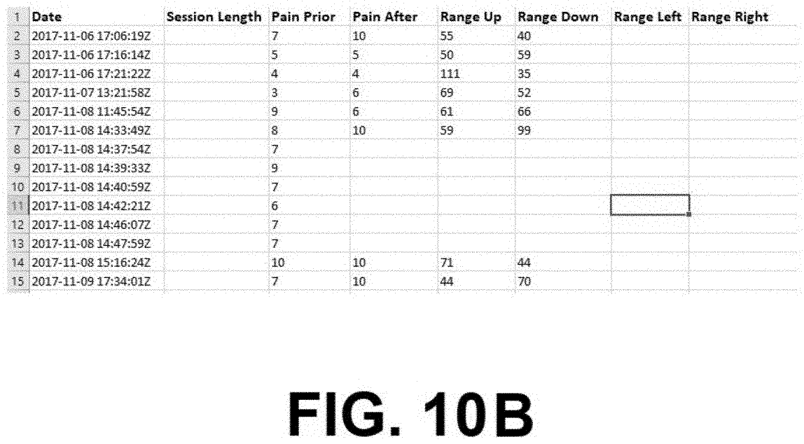

FIGS. 10A and 10B show example non-limiting GUI screen captures of potential ways of displaying patient data, either to a patient or to a physical therapist.

The patient portal can contain, for example, inquiries as to skills, resources, various elements to aid inpatient/outpatient rehabilitation, and other things. When a person clicks a “learn more” button (a type of call to action button), that person is prompted to enter their first name, last name, email address, mechanisms for the administrators of the system 100 to contact that person.

In an embodiment, some features of the system 100 include weekly goals, notification reminders, and the accountability buddy 522. This would be a person known and trusted by the patient, where if that person becomes aware a patient is blowing off their therapy, someone that a patient trusts, but not just the Physical Therapist, who will give a reminder, e.g. “Do your therapy”! An example accountability buddy 522 is shown in FIG. 5B.

The patient portal shows a real-time messaging, also direct communication with therapists, a patient's game allotments, motions, and pain levels, full game suite, other things.

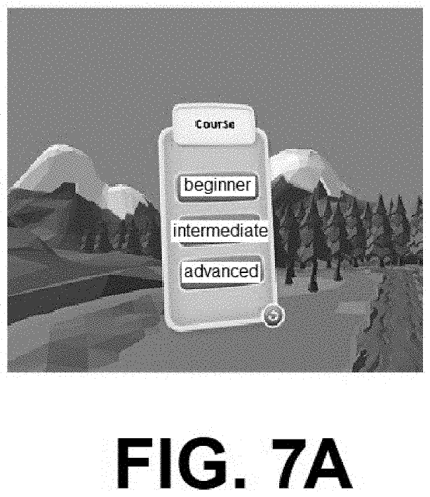

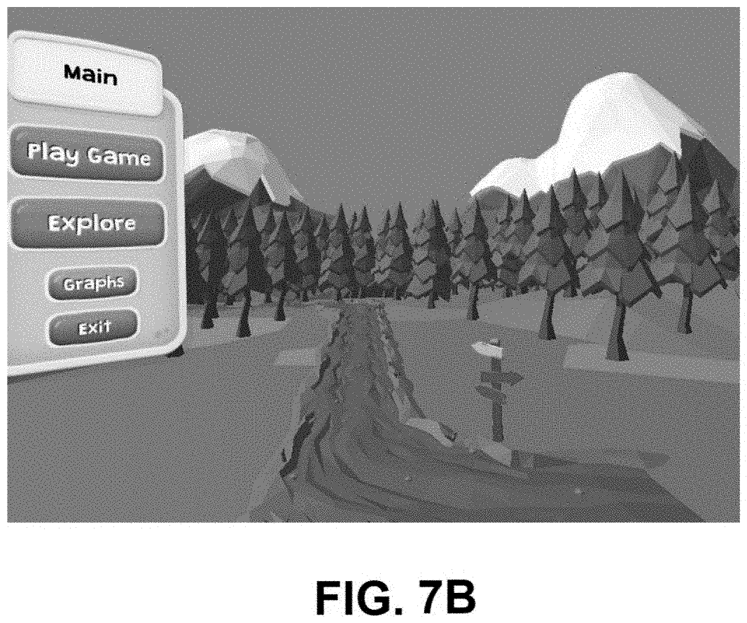

FIG. 7A shows an example, non-limiting GUI presented to a potential patient, showing how a game can have three options: beginner, intermediate, advanced. However, the embodiments herein should not be considered limited to this scenario, as embodiments of the system 100 exist in which game-difficulty levels are systematically adjusted based on individual patient's progress. For example, FIG. 9A shows a VR GUI 902 showing a patient progress chart 942 (left side) based on that patient's achievements, aka points and game levels they have reached. Thus, the three-option embodiment shown in FIG. 7A would not always be employed, instead the system 100 might compute and set game-difficulty levels without need for patient interaction. However, the option shown in FIG. 7A could still be available, such as in a patient-override (patient choice) situation.

The system 100 employs machine learning or artificial intelligence technology. Therapists need to understand how different therapies interact with the patient, including different injuries, age groups, and gender. The analysis will help therapists build intelligent exercise therapies based on patient data. For example, if the patient is a white male between 30 and 40 years of age with a shoulder spur, the system 100 can provide a list of the most effective exercises based on data analysis. Other age groups or other demographics may require different exercises for the same or less severe injury.

FIG. 7B shows an example GUI displaying various options to a patient, comprising “play game,” “explore,” “graphs,” and “exit”. The “play game” option could lead the patient to e.g. the GUIs shown in FIGS. 9A or 9C. The “graphs” option could bring a patient to the GUIs within e.g. FIGS. 10A-10B, 13A-13B, or 14C-14E.

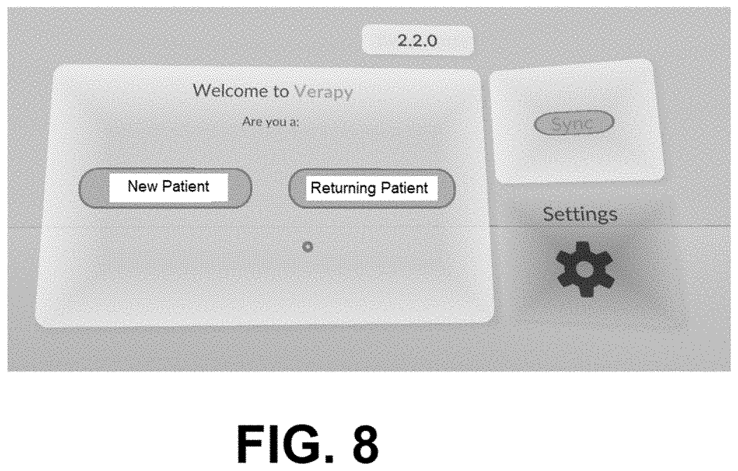

FIG. 8 shows an example, non-limiting GUI for a main menu that either a new patient or an existing patient may encounter when logging into the system 100. This is where the patient can opt in to review the tutorial if they have missed couple of session and may need a quick refresher. Otherwise, they will click on “Existing patient” to resume their normal therapy sessions. For extra security, the user must input a PIN every time they log into the system 100.

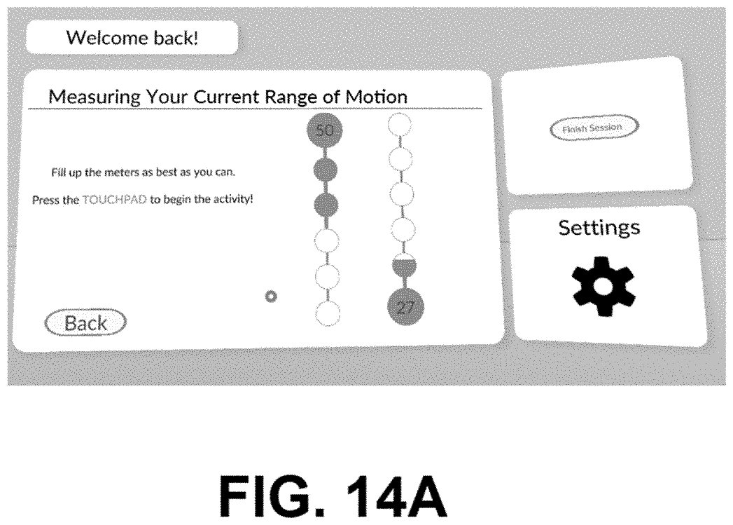

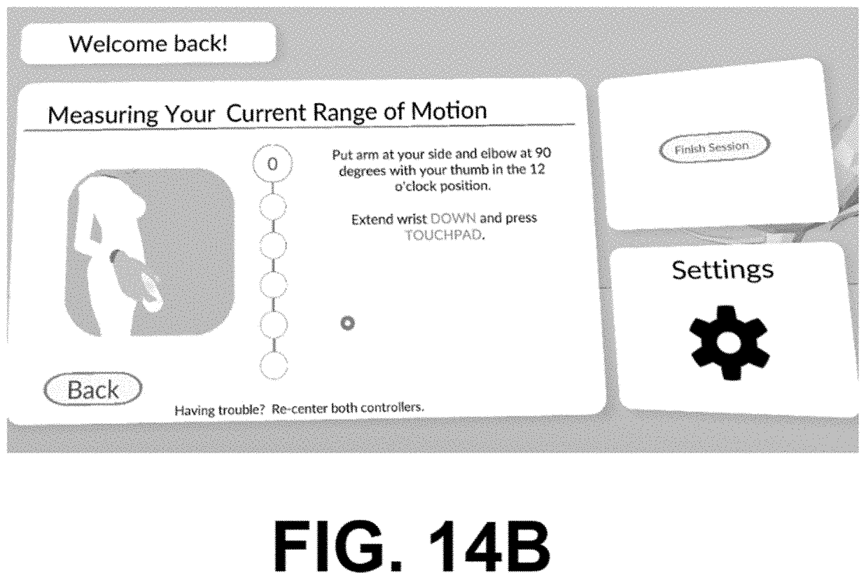

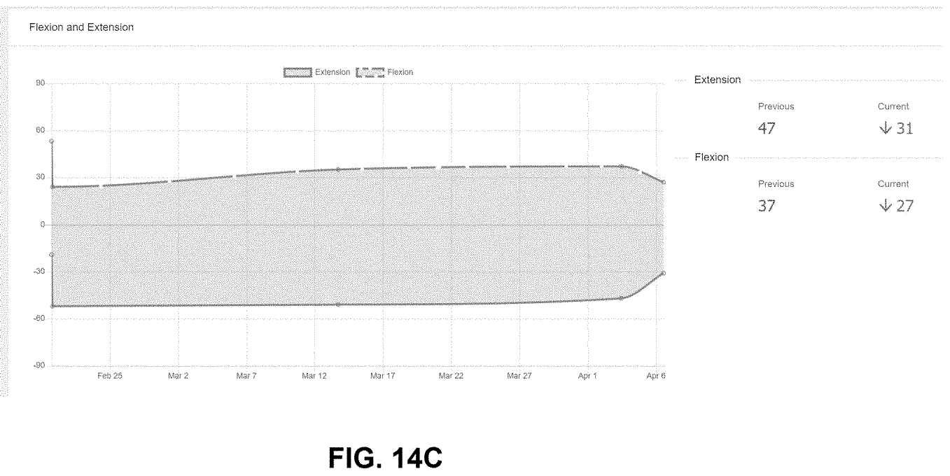

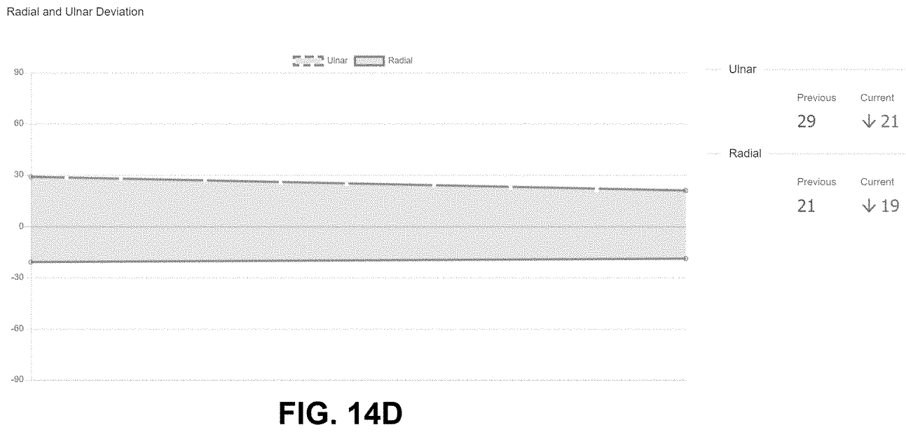

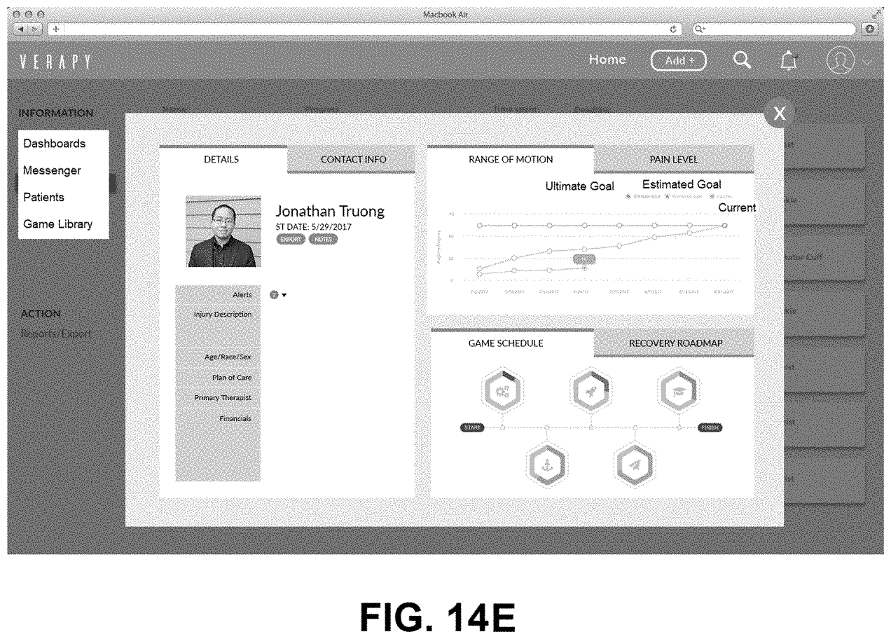

FIGS. 14A and 14B show example, non-limiting GUIs for obtaining range-of-motion levels from patients, while FIGS. 14C, 14D, and 14E show example, non-limiting GUIs for displaying those range-of-motion levels. From FIGS. 14A-E it is apparent that the range-of-motion GUI tracks current, estimated goal, and ultimate goal for range of motion to be achieved. Regarding the current range of motion, it is important to perform per-person calibration. Such per-person pre-PT calibration means that before patients do the therapy, the therapist will calibrate or take measurements of that patient's current passive range of motion. Such calibration would determine what the existing range of motion is, where a patient might say “I can only do so much, this is my starting point”. Conversely, a one-size-fits-all non-calibrated range-metrics could be misleading. Customization is better.

For example, one possible range of motion for an arm or shoulder might be e.g. 45° up and 45° down. However, an injured person may not have that range of motion at their beginning of starting a PT regimen. Some injured persons may only have range of 15° up and 15° down. In such a case, a PT regimen eventually improving that person to 20° up and 20° down would be considered an improvement. However, a rigid and inflexible metric like “all patients must be improved to e.g. 45° up and 45° down” could serve to potentially mis-characterize a PT regimen as non-compliant or ineffective, when in fact it is effective.

Before each game session, the system 100 will automatically capture the specific metrics for each patient. By following the prompts shown in the GUIs of FIGS. 14A and 14B, patients will perform an initial range test using an avatar. The instruction visually and in writing also help to make sure that the measurement of range of motion is taken accurately and correctly.

Another way to look at it, measuring how much someone improves must involve knowing where they started at in the first place. This is true for range-of-motion issues, but also true for patient progress in general. The difficulty levels going forward are systematically adjusted based on individual patient's progress. For example, In FIG. 9A on the left side of the patient-GUI shows a patient progress chart 942 based on their achievements, aka points and game levels they have reached.

FIG. 2B shows how the system processes patient choices in setting up the various games and accommodating the patient's choices. The flowchart of FIG. 2B is a deep dive from the diagram shown in FIG. 2A, specifically as relates to “Session Data” and “Session Configuration”, and the whole workflow function.

FIG. 9A shows an example-only, non-limiting GUI of potential body-areas needing PT/OT, so that the system 100 can make a decision on which types of games to offer to the patient, that is, which games would be suitable for that specific body-area. FIG. 2B shows more information about how the system 100 goes through the decision-making process. The body-area selection window GUI of FIG. 9A comprises a display of various body parts and the PT exercises that are available through the system 100. Typically, a patient would only see what is applicable to their specific injury or condition. For example, for a post op total knee replacement patient, there will be only knee exercises visible/available. When each unique patient profile is entered into the system, the therapist would select patient injury type and the body part as a way to determine a specific plan of care.

FIG. 9B shows an example-only, non-limiting lists of various PT/OT games, while FIGS. 9D and 9E show GUI screen captures of potential, non-limiting game-environments as seen by a patient.

FIG. 9F shows what game-GUI would appear to a patient supposing the patient selected the sand-castle game from the GUI of FIG. 9C. Within FIG. 9F, a patient playing the sand-castle game would operate the hammer 920 to pound the sandcastle 924.

When a patient puts on the VR headset 306, that patient may see the GUI shown in FIG. 7B, e.g. a mountain range, forest, ambient music and see “main menu” to start playing a game. However, other GUIs may be displayed instead, so that FIG. 7B may not be displayed at all.

FIG. 9D shows an example ring-grab game. In order for this specification to be fully-enabling, it is necessary to at least attempt to explain some of the games in a prose context, since human movement is involved yet video cannot be included in a patent application. Within the ring-grab game shown in FIG. 9D, a patient navigates the various rings 928 using a bird 924 as an avatar. In some cases, coins are positioned above and below the rings 928. The avatar\bird 924 will attempt to catch the rings 928, where that bird's movements are associated with the movements of a patient during therapy. Within this particular game, the assumption is that a patient will move an injured arm up and down (flexion and extension movement) to try to grasp the rings. However, the embodiments herein are not limited solely to this type of game or this type of motion.

As the patient is playing this therapy game, or many of the others, that patient will see a red numbers (not visible in FIG. 9D) at the bottom right corner indicating the amount of range of motion in real-time. When that patient reaches towards the end of the game, the system 100 will show the results of rings captured and coins earned.

After finishing the game, a variety of GUIs can show the amount of range of motion acquired, of which only one non-limiting example is shown in FIG. 10A and 10B as measured by the wearable (body attachment) 304 along with the sensor controller 320. Further, FIG. 10A file shows detailed information about range-of-motion data. As shown in FIG. 10B, some example .csv data is displayed in rows and columns capturing the range of motion by a number judgment each second, all real time, so if a 30-minute session was experienced, all data from the entire session is available. The system 100 can display this data in a variety of ways, and is not limited solely to what is shown within FIGS. 10A and 10B, which are provided for illustration and example only. For example, range-of-motion data is also the subject of FIGS. 14C-14E.

In conventional PT/OT environments, a patient's range of motion is very subjective much manually, eyeballing, “hey I can see you are at about 35 degrees or 45 degrees” which is subjective and not always accurate. Meanwhile, using the wearables (body attachments) 304 and the sensor controller 320 described herein; the system 100 can have e.g. 97.5% accuracy, as shown for example in FIGS. 10A-10B.

### Begin Therapist Portal

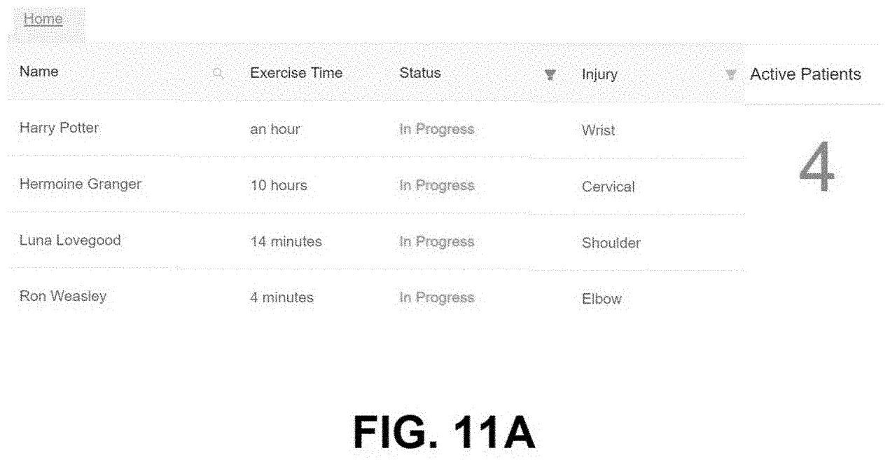

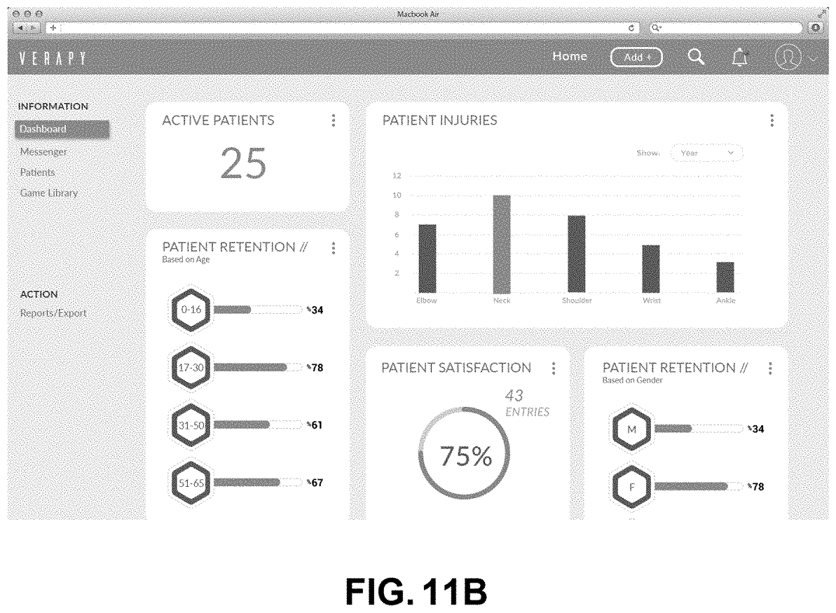

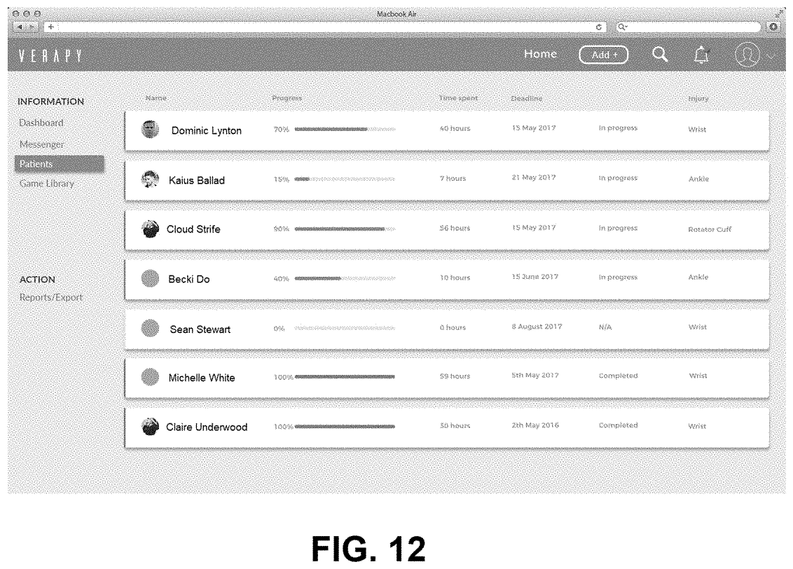

Moving on from the patient portal, some example GUIs within a Physical Therapist portal will now be discussed. FIGS. 11A, 11B, and 12 show example, non-limiting GUIs of what Physical Therapists would see, including example patients and their specific conditions (FIG. 11A), and an overall dashboard-view (FIG. 11B). FIG. 12 shows a different type of GUI, but similar, displaying a list of patients with some of their conditions displayed in columns. Further information is available by clicking on the specific patient. The example GUIs of FIGS. 11A, 11B, and 12 are non-limiting and are provided for illustration and example only.

As stated, FIGS. 10A and 10B show example non-limiting GUI screen captures of potential ways of displaying patient data, either to a patient or to a physical therapist.

### Tutorials

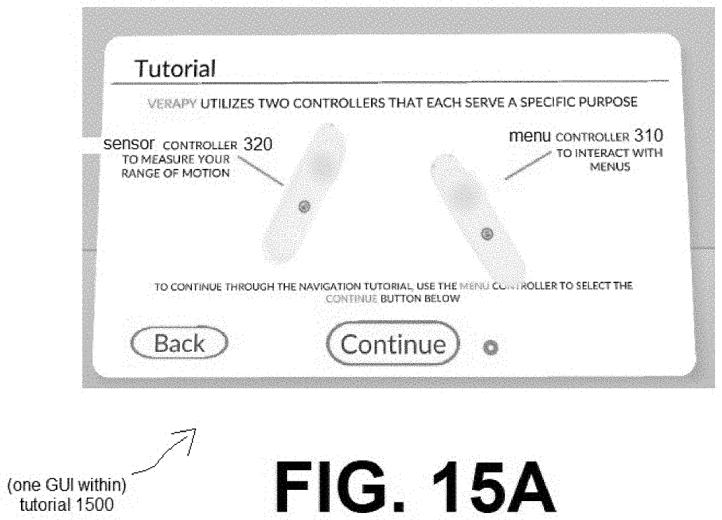

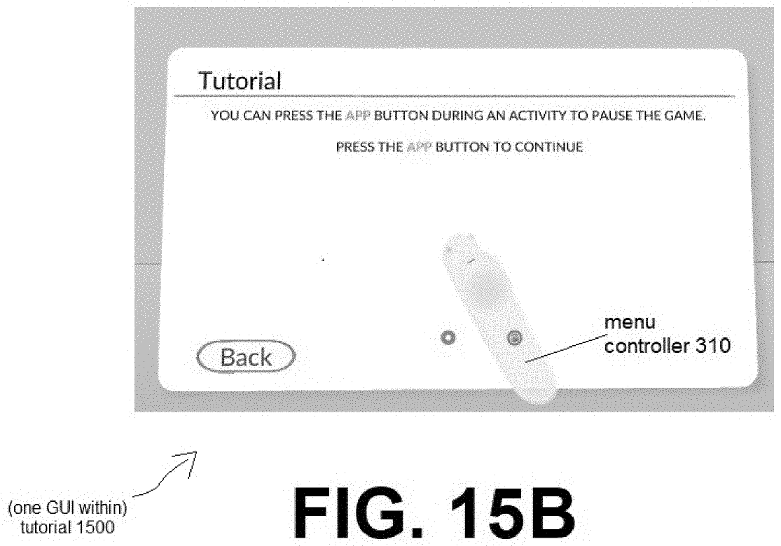

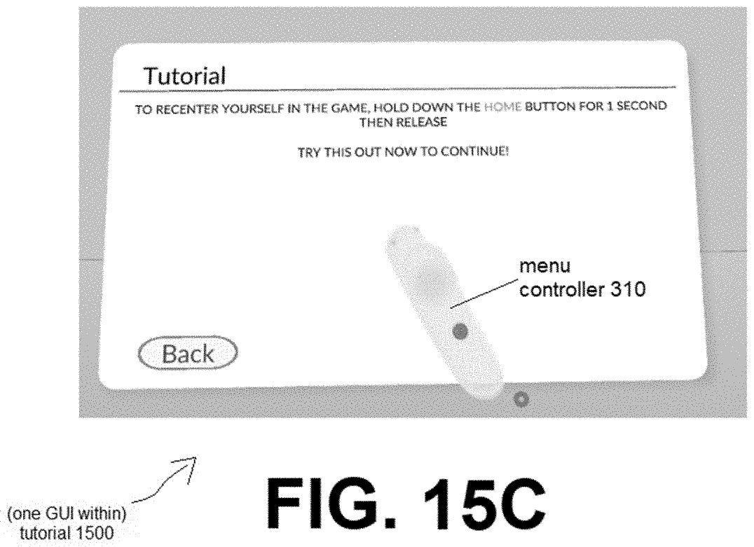

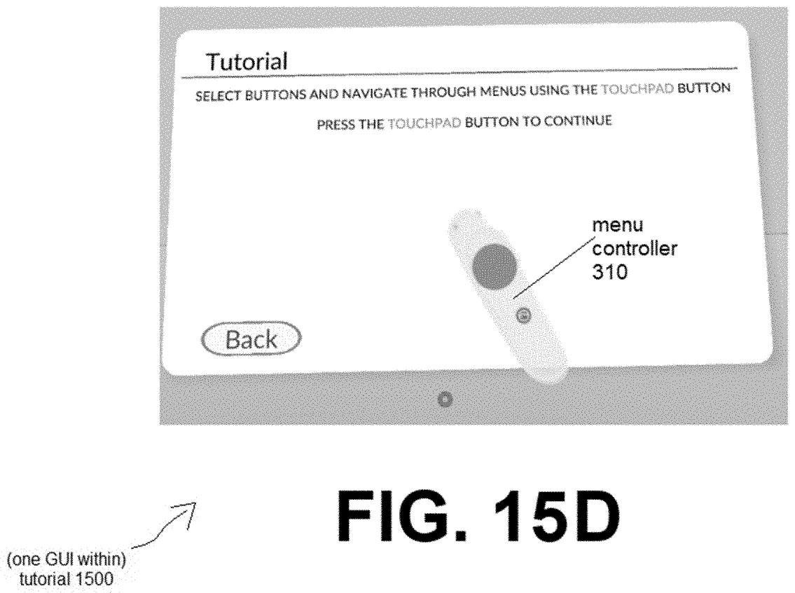

FIGS. 15A, 15B, 15C, and 15D show example, non-limiting GUIs for patient-tutorials showing patients how to use the embodiments herein. A majority of patients are new to virtual reality technology, let alone have experienced the immersive nature of it. In order to provide seamless patient engagement, the embodiments herein provide a short tutorial where patients are familiarized with the system 100 in terms of navigation, usability and differentiation between the menu controller 310 and the sensor controller 320. Interactive cues walk the patients through simple and quick steps before they are ready to engage into the therapy session.

An “exercise preview tutorial” also is part of the tutorial, and exists to help the patient see (through the avatar) what a specific exercise and its associated movement looks like. It is well-known that patients learn their exercises by watching how the Physical Therapist demonstrates that exercise, so that some sort of visual flow can be invaluable. It is important to make sure that patients follow the prescribed therapy at times when the Physical Therapist may be pre-occupied or hands on with other patients. Such an exercise preview tutorial can be invaluable in achieving this, thus saving considerable time and patience.

### Error-Checking and Usability

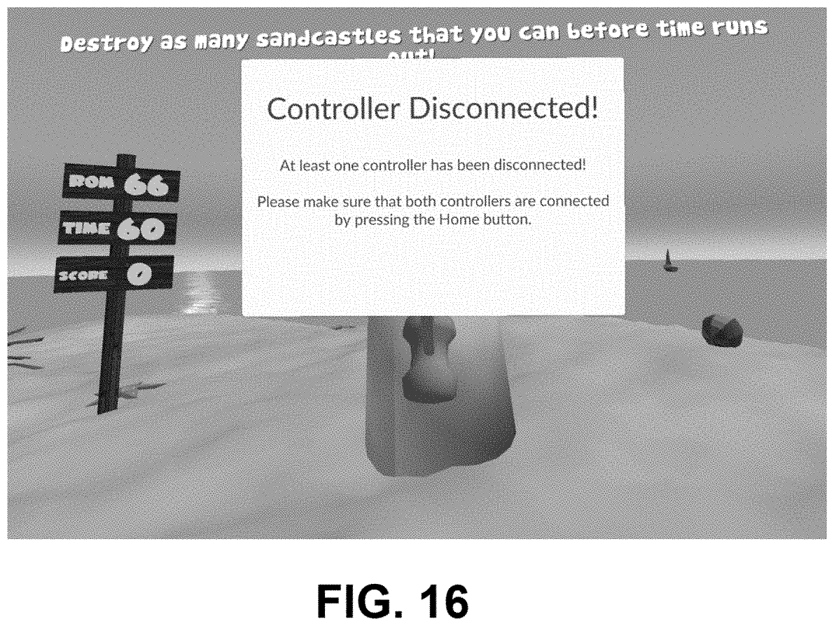

There are a lot of moving parts and adjustable aspects of the system 100, which is very configurable. As such, there are also a lot of ways for things to go wrong, for software to act buggy, or for some type of patient-misunderstanding. The embodiments herein take several steps to minimize any such errors, including frequent polling to make sure all components are properly connected. For example, FIG. 16 shows an example, non-limiting GUI for alerting a patient to a potential error or disconnection of a sensor. When using the sensor controller 320, if for whatever reason the connection is disrupted, the system 100 will auto-notify the patient to check the controller for connectivity as the session data would not get recorded without it working. This is to prevent any loss of data during patient's therapy session.

### Expanding the Business Model

The system 100 embraces not just PT, but also occupational therapy that could ultimately use the system 100. For example, chiropractors, VA hospitals (veterans with PTSD), acute patients, outpatient care centers, cancer patients and others could use the system 100. Could be something used for traumatic brain injury with the reality of relearning certain things neurologically, to overcome for example the effects of a concussion.

Other embodiments can also include Biomechanical chemical analysis and mechanical retraining. Could be used for some “work hardening.” This means, using an example of e.g. an agricultural worker, on a farm. A ditch digger who has to shovel and to ditch every day and to change the way to dig the ditch, the mechanics of pointing the shovel, picking up the dirt, scooping, dumping the dirt out of the shovel, etc., thus, a concept of “work hardening.”

Another issue addressed by the embodiments disclosed herein is potential for fraud and abuse. There exist some workmen's compensation claimants who are incentivized not to get better, because they get paid two-thirds of their regular salary while they are unable to work. They receive temporary total disability. To prevent fraud, the system 100 can obtain data about what the patients are asserting. Data on their compliance could be useful as far as showing that the doctor whether they are malingering to defend the comp claim.

Also, because there's more into value-based care, the physical therapists or all health care providers will be penalized in the cases where the patient is non-compliant. The insurance companies may demand penalties back from them. In reverse, every patient that is compliant and that does the therapy whatever the health care was prescribed; they may get incentives from the payers. This practice has already started but is going to evolve and be fully implemented over the next few years.

Next, there is a certain mindset among insurance outcomes sessions: seeing who were the ones that get the better outcomes and giving bonuses for improving outcomes. The system 100 can provide metrics for insurance carriers to the same therapists. Only have a network of physical therapists known to get the right outcomes, the system 100 would give them analytical data to show which therapists weren't getting the desired outcomes by systematically analyzing a number of patients over certain time interval. The system 100 could be used by them to cherry pick and have the best provider-network available so that the insurance companies are saving money by sending their patients or their insureds, to the therapists having the best outcomes.

Now let's say a good therapist has a dud, a recalcitrant patient that just will not do his/her home routines, just won't do it. Using the system 100, data is available to show that the patient, not the therapist, was the problem. This would help to prevent earnest, effective therapists from being dropped.

All physical therapists will have some patients that can get better or the injuries such that can't get better. But when looking amongst everyone else cases or a similar sample population, the same number of superstars, while one can look at the providers that seem to have more skewed towards one side or the other because that's what will distinguish the quality therapist.

Using the system 100, it is possible to prove that the patients are doing the exercises or that their specific injury or condition is not responding well to treatment.

To identify patient upon a login, a biometric, retinal and similar recognition technology will make sure to provide access to the correct patient, thus reducing data fraud which will make insurance companies a bit more interested, based on HIPAA compliance, etc.

FIGS. 11A, 11B, 12, 13A, and 13B also show information useful to a therapist, including for example FIG. 12, which shows GUIs tracking patient progress, amount of time spent, and any deadlines that may exist. This may include insurance-related deadlines, workman's comp deadlines, or other deadlines.

Similarly, the dashboards shown in FIGS. 11A-11B could be useful for showing the overall effectiveness of either a single therapist, or aggregating several therapists and showing the overall effectiveness of an entire PT office.

### Crypto-Rewards

After playing a game, and earning certain types of rewards for effort, it may be possible to redeem those rewards in some type of cryptocurrency or crypto-coin context. In 2019 and beyond, there is huge fascination in the general public, the pool of potential patients, with cryptocurrency concepts.

### Analysis\Value of the Embodiments Described Herein

Going back to the economic benefits compliance is a problem. People are not motivated to do their physical therapy. Reinjury and re-admittance cost not only insurance companies, but us as all participants in the health care system. Improving compliance also reduces reinjuries which subsequently lowers re-admittance rates. When patients are re-admitted, that comes out of the pockets of all payers, especially health insurance because notes with therapy for more time, hence more money spent. By gamifying the PT process, patients are more motivated, thus improving retention and ultimately the outcomes.

As insurance company must pay more, those costs are spread out as higher premiums throughout all policyholders. For example, suppose an employee of company X hurt their shoulder and have several recurrences and therefore more therapy. Now let's say the therapy program cost the company an additional $10,000, and the company employs 10,000 people. If so, everyone pays an additional one dollar premium the following year. This is because premiums are based on, or at least factor in, loss history to a certain extent. As such, the system 100 reduces costs by improving outcomes, improving outcomes via better compliance, and improving compliance by making the PT more fun and interesting to participate in.

The following analysis comes from the PoV of Therapists. Inside a PT office, assume a similar staff is getting to see patients, there are usually two chairs that listen to patients at one therapist can cater to both patients. Using the system 100, that same therapist can see into e.g. three patients, because all three are sitting down with the VR environment 300 and doing therapy. That improves efficiency, avoids an increase in the staff, and gets them increasing the number of patients being serviced, but without reducing the value of care.

This also becomes a reimbursable expense by the insurance carriers under the existing CPT billing codes which are five digit codes to denote particular services. Accordingly, this is eventually something that would be an additional service such as virtual reality modality just like an ice pack or heat pack or electro-stimulation, or group exercise or therapy that this would be something that they would put on the HIPAA form that goes to the insurance carriers, which recognize that code and have a reimbursable service back to the physical therapist.

As such, with the value based care model that healthcare is moving toward, incorporating telehealth and telemedicine, PT is one of the top use-cases being considered for reimbursement by e.g. Medicare, insurance companies, etc. Utilizing virtual reality physical rehabilitation will enable physical therapy clinics to incorporate telehealth services to those patients that are home-bound or less likely to travel to the PT clinic.

An insurance company will see the benefit because, if they can enable system 100, they can get better outcomes with fewer visits. This is superior to just having a certain number of visits and then putting someone in a “home” program, and never having certainty patients are doing it. Instead, the system 100 provides a more engaging home program and enables tracking of metrics and activity, and therefore those few visits would be far more effective and could be used more efficiently.

Although stated earlier, it is worth repeating that people not doing their home exercises programs is a big problem and cost-drain in the healthcare industry. Even motivated people blow it off other than go to the PT clinic.

### Disclaimer

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations, or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

### B-Team Backup Materials

The following material, in the form of claims, are included herein as part of the Specification and not part of the official listing of claims.

2. The method of claim 1, further comprising:
the website and access portal software uploading the session configuration to the internet-enabled VR therapy arrangement.

11. The method of claim 10, further comprising:
the physical therapist portal displaying medications prescribed, types of injury, and patients information cross-coordinated with specific games and injury areas.

13B. The method of claim 1, further comprising:
configuring the physical therapy games with difficulty levels that are adjusted by an algorithm contained within the virtual service component.

14. The method of claim 1, further comprising:
tapping into a patient's pre-existing enjoyment of video games.

15. The method of claim 1, further comprising:
increasing patient empowerment through the patients seeing their results displayed in a objective graphical-numeric format and also increased participation of continuing the therapy day in and day out without lapses.

16. The method of claim 1, further comprising:
configuring the website and access portal to include an ultimate goal signifying where that patient can consider themselves to be fully restored to optimal condition as they were before the incident or problem that necessitated their physical therapy.

17. The method of claim 1, further comprising:
configuring the website and access portal to include an interval goal where patients want to be at a certain time, marked by progress points at a certain time in the future.

18. The method of claim 1, further comprising:
configuring the website and access portal to include a current status explaining where that patient exists currently.

19. The method of claim 1, further comprising:
a physical therapist mapping out reasonable progress goals e.g. 50% range of motion; the next week get 75% range of motion, then 100% range of motion. Another checkpoint might be a further increase in the range of motion combined with 25% strength.

What is claimed is:

1. A method for configuring and providing a physical therapy system, comprising:
positioning and facilitating a website and access portal, an arrangement of servers and hosting products, an arrangement of game servers, a game development environment, a patient home environment, and a Physical Therapy (PT) office environment to all be connected with each other through a computer network;
configuring a patient home environment with a least one arrangement of Virtual Reality (VR) equipment;
configuring a PT office environment with a plurality of arrangements of VR equipment;
a physical therapist selecting a plan of treatment for a specific patient using a secure web interface hosted on a virtual service component; and
the virtual service component converting the plan of treatment into a session configuration comprising one or more PT games for that specific patient.

2. The method of claim 1, the internet-enabled VR therapy arrangement comprising a VR goggles, a body attachment, and a sensor controller.

3. The method of claim 1, further comprising:
directly acquiring pain level data from the patient in the form of questionnaires.

4. The method of claim 1, further comprising:
indirectly acquiring range of motion data from the patient based on sensor readings from the internet-enabled VR therapy arrangement.

5. The method of claim 2, further comprising:
the internet-enabled VR therapy arrangement uploading data recorded from a patient's therapy session (referred to hereinafter as session data) to the virtual service component.

6. The method of claim 1, further comprising:
the virtual service component generating reports, charts, and graphs for each patient based on that patient's session data, thereby
providing feedback about a patient's therapeutic progress.

7. The method of claim 6, further comprising:
posting the patient therapeutic progress information into the website and access portal to be available to both the patient and the physical therapist.

8. The method of claim 1, further comprising:
tracking patient progress milestones including but not limited to completion of each session from the plan of treatment on schedule; wherein
if progress is not satisfactory, or if sessions from the plan of treatment are missed, sending notifications to the patient and an accountability contact of the patient.

9. The method of claim 1, further comprising:
providing both information and supervisory portals comprising a patient portal, a physical therapist portal, and an administrator portal.

10. The method of claim 9, further comprising:
the physical therapist portal displaying patients grouped by specific games, such that different patients with different disabilities/injuries are associated with a game that is appropriate for that disability or injury.

11. The method of claim 1, further comprising:
as part of patients playing the physical therapy games, collecting and unlocking achievements by that patient connected to some measurable phase of their physical therapy.

12. The method of claim 1, further comprising:
before starting a game, a patient GUI prompting a patient to provide their pain level on scale 1 to 10;
when a patient completes the game, the patient GUI prompting that patient for their post-game pain level from 1 to 10;
storing and mapping the pain levels in a type of time-mapped progression, week over week, month over month; and
making the time-mapped progressions available to the patient and to the physical therapist.

13. The method of claim 1, further comprising:
configuring the physical therapy games with difficulty levels that are adjusted by therapist based on a professional treatment plan.

14. The method of claim 1, further comprising:
gamifying the PT process such that patients are more motivated to participate, thus improving the outcomes, thereby
reducing re-injury and re-admittances thus reducing insurance costs.

15. The method of claim 1, further comprising:
a single therapist simultaneously supervising the PT activities of a plurality of patients simultaneously thereby improving efficiency, avoiding an increase in staff, and increasing the number of patients being serviced but without reducing the quality of care.

16. The method of claim 1, further comprising:
by providing a more engaging home program and enabling tracking of metrics thereby reducing PT visits, an insurance company seeing improvements in patient outcomes;
the remaining PT visits increasing in effectiveness and efficiency.

17. The method of claim 1, further comprising:
reducing incidences of workmen's compensation claimants who are incentivized to not get better, by
obtaining accurate compliance data about patient-participation, thereby
helping to show existence of malingering solely for the purpose of prolonging the workman's compensation claim; thereby
reducing potential for fraud and abuse.

18. The method of claim 1, further comprising:
providing metrics for insurance carriers about therapists;
an insurance company utilizing data obtained by the website and access portal to determine and rank which therapists obtaining better outcomes and giving bonuses for improving outcomes;
restricting a network of physical therapists that are known to get the right outcomes by utilizing analytical data;
discharging therapists who aren't getting the desired outcomes.

19. The method of claim 18, further comprising:
protecting competent physical therapists working with uncooperative patients that just will not do their home routines;
providing data showing that the patient, not the therapist, was the problem; thereby
preventing earnest, effective therapists from being dropped from lists of qualified providers due to misleading or incomplete patient-outcome statistics.

20. The method of claim 1, further comprising:
increasing accuracy in determining whether patients are truly completing their assigned PT tasks.
